(19) United States
(12) Patent Application Publication
(10) Pub. No.: US 2005/0192658 A1
(43) Pub. Date: Sep. 1, 2005

(54) Apparatus and method for the articulation of a catheter

(76) Inventors: Terry Dahl, Santa Barbara, CA (US); Stan Needle, Louisville, CO (US); Hugh H. Trout III, Bethesda, MD (US)

Correspondence Address: COLLIER SHANNON SCOTT, PLLC, 3050 K STREET, NW, SUITE 400, WASHINGTON, DC, 20007, US

(21) Appl. No.: 11/038,407

(22) Filed: Jan. 21, 2005

Related U.S. Application Data
(60) Provisional application No. 60/538,242, filed on Jan. 23, 2004.

(51) Int. Cl. A61F 2/06

(52) U.S. Cl. 623/1.11

(57) ABSTRACT

A delivery apparatus for delivering a surgical device comprising, a catheter having a distal segment, a proximate segment, and a lumen. The distal segment has a first configuration having a curved shape. A delivery component is then inserted within the lumen of the catheter where the delivery component has a first and a second segment wherein the first segment has rigidity greater than the catheter and the second segment having rigidity less than the catheter. The distal segment of the catheter is bent straight into a second configuration by the first segment of the delivery component that is more rigid than the catheter when that segment is moved along the entire distal end of the catheter. The catheter can then bend into a third configuration that is curved when the delivery component is moved so that a second segment of the delivery component comes into contact with lumen of the distal end of the catheter where the distal end of the catheter is more rigid than the second segment of the delivery component.

### CROSS-REFERENCE TO RELATED APPLICATIONS

The present invention relates to, and is entitled to the benefit of the earlier filing date and priority of, Application No. 60/538,242 filed on Jan. 23, 2004.

### FIELD OF THE INVENTION

The present invention relates generally an apparatus for the articulation of a catheter and method for use in surgical repair.

### BACKGROUND

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm as no statistical benefit exists to do so.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is extremely high, there is also considerable mortality and morbidity associated with open surgical intervention to repair an aortic aneurysm. This intervention involves penetrating the abdominal wall to the location of the aneurysm to reinforce or replace the diseased section of the abdominal wall (i.e., abdominal aorta). A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an abdominal aortic aneurysm without the attendant risks of intra-abdominal surgical intervention. Although techniques have been developed that may reduce the stress, morbidity, and risk of mortality associated with surgical intervention to repair aortic aneurysms, none of the prior art systems that have been developed effectively treat the aneurysm and exclude the affected section of aorta from the pressures and stresses associated with circulation. None of the devices disclosed in the references provide a reliable and quick means to reinforce an aneurysmal artery. In addition, all of the prior references require a sufficiently large section of healthy aorta abutting the aneurysm to ensure attachment of the graft. The proximal aortic neck (i.e., above the aneurysm) is usually sufficient to support a graft's attachment means. However, when an aneurysm is located near the iliac arteries, there may be an ill-defined neck or no neck below the aneurysm. Such an ill-defined neck would have an insufficient amount of healthy aortic tissue to which to successfully attach a graft. Furthermore, much of the abdominal aortic wall may be calcified making it extremely difficult to attach a graft thereto.

One of the problems with treating this type of aneurysm is that a catheter that is capable of articulation and assuming a bent configuration is often needed to manipulate or attach a graft or perform some type of surgical function. Catheters that have “preset” curves are difficult to work with because the catheter must first be advanced to the surgical location. Other existing methods of articulating catheters involve using guide wires to bend the catheter, but this type of catheter has also been problematic because it often requires the removal of the guide wire to allow the catheter to bend.

Thus, there is a need in the industry for an improved method and apparatus for articulating catheters to be curved or bent during the deployment of the catheter.

Additional advantages of various embodiments of the invention are set forth, in part, in the description that follows and, in part, will be apparent to those of ordinary skill in the art from the description and/or from the practice of the invention. It is an advantage of an embodiment of the present invention to provide a method and apparatus for curving or bending a distal end of a catheter while the catheter is inside the body. It is an additional advantage of an embodiment of the present invention to provide a catheter for deploying surgical components within the vascular system to assist in the repair of abdominal aortic aneurysms.

### SUMMARY

An embodiment of the present invention provides an apparatus and a method for allowing the articulation of a catheter having a distal end and a lumen wherein the distal end of the catheter has a preformed bend. A delivery component with at least two segments of different rigidities is inserted into the catheter so that one segment that is more rigid than the distal end of the catheter straightens the distal end of the catheter. The delivery component can then be moved so that a second segment that is not as rigid as the distal end of the catheter comes into contact with the distal end allowing the distal end of the catheter to bend or assume all or a portion of the preformed configuration to curve.

Additional advantages and novel features of embodiments of the invention will be set forth in the following description and will become apparent to those skilled in the art upon reading this description or practicing the invention. The advantages of embodiments of the invention may be realized and attained by the appended claims.

### BRIEF DESCRIPTION OF THE DRAWINGS

The accompanying drawings, which are incorporated in and constitute a part of this specification, illustrate embodiments of the invention and together with the description, serve to explain the principles of the invention. Where appropriate, the same reference numerals refer to the same or similar elements.

FIG. 1 is a schematic view of a flexible guide wire.

FIG. 2 is a schematic view of a catheter.

FIG. 3 is a schematic view of an artery with a guide wire and a catheter.

FIG. 4 is a schematic view of an artery with a catheter.

FIG. 5 is a schematic view of a delivery wire with a delivery device according to an embodiment of the present invention.

FIG. 6 is a schematic view of a catheter according to an embodiment of the present invention.

FIGS. 7, 8 and 9 are schematic views of the delivery wire and catheter according to an embodiment of the present invention.

FIGS. 10, 11 and 12 are schematic views of the catheter according to an embodiment of the present invention inserted through an artery into the aneurismal portion of the aorta.

FIGS. 13 and 14 are schematic views of a support device attached to a prosthetic graft.

### DETAILED DESCRIPTION OF EMBODIMENTS OF THE INVENTION

Reference now will be made in detail to the apparatus and methods consistent with implementations of the present invention, examples of which are illustrated in the accompanying drawings. The appended claims define the scope of the invention, and the following description does not limit that scope.

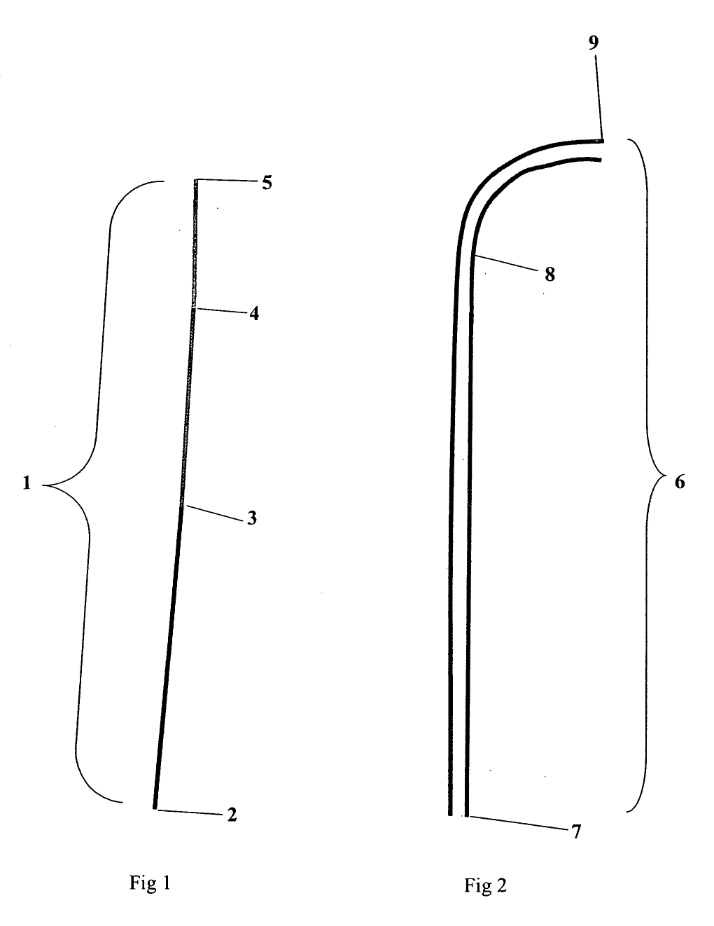

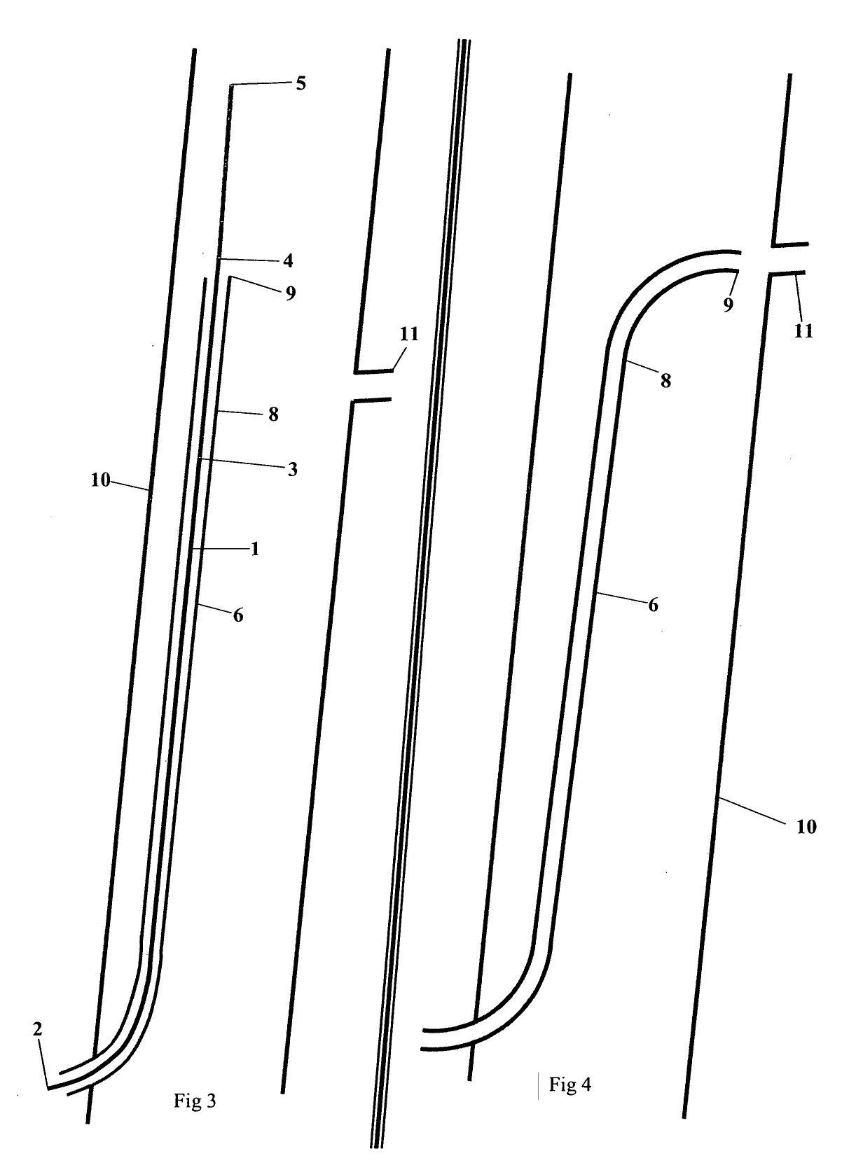

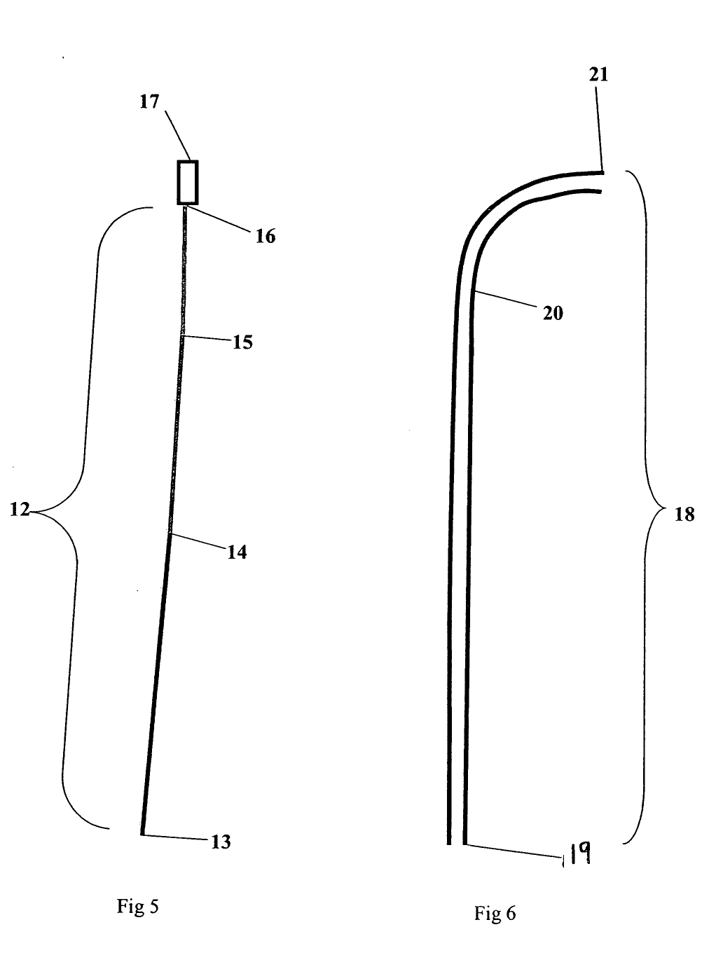

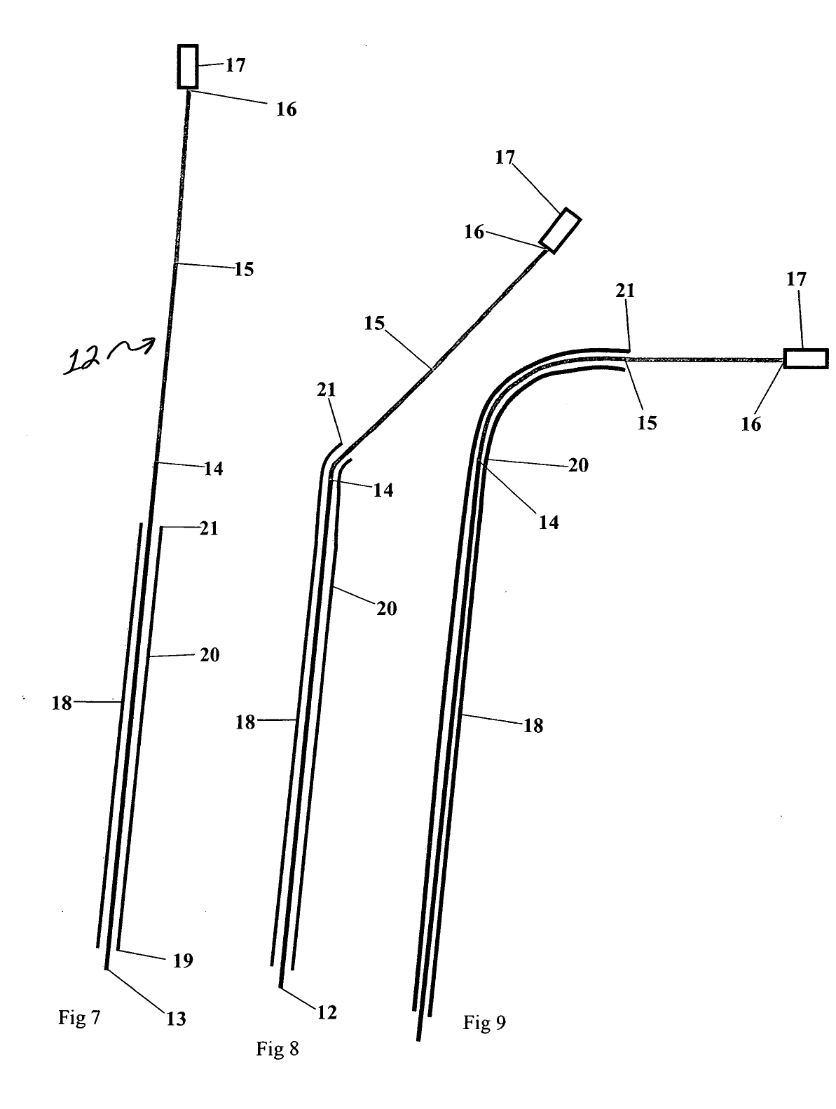

As shown in FIG. 1, current practice for inserting a catheter into a lumen, such as a a blood vessel, is to take guide wire 1 composed of one or more segments such as 2-3, 3-4, and 4-5 of varying flexibility, usually progressing from most flexible at the leading component 4-5 to the least flexible at the trailing component 2-3. The catheter 6 can be straight but often it has a different configuration at its leading edge such as shown in FIG. 2 with a straight component 7-8 leading to a curved portion 8-9, at the leading edge. One of the purposes of this approach is to allow catheter 6 with a specially designed end, such as a curve, to be inserted into a vessel so that the operator will subsequently be able to insert guide wire 1 into an otherwise inaccessible vessel lumen. FIG. 3 depicts an artery 10 with guidewire 1 within its lumen and catheter 6 inserted over guidewire 1 at portions 2-3-4-5. Note that the different catheter 6 configuration, in this instance a curved portion 8-9 is held straight by the stiffness of guidewire 1. As shown in FIG. 4, once the guidewire is removed, catheter 6 resumes its preformed configuration at its leading edge, in this instance curve 8-9 with the catheter 6 tip positioned near the orifice of a branch 11 of the vessel.

Embodiments according to the present invention and method described below are novel from those described above. In the approach above, catheter 6 is inserted into a vessel over a guide wire 1 and allowed to resume its shape only when the guide wire 1 is removed. In the embodiments according to the present invention and method described below, the catheter 6 is distracted from its configured shape for a portion of its insertion over delivery apparatus 12, in this instance may be a wire (but it could be a plastic rod, round, oblong or with an edge or a metal alloy tube), but catheter 6 configuration then is used to bend delivery apparatus 12 to facilitate insertion or maneuvering of a surgical device 17 attached to the delivery apparatus 12 within a vessel.

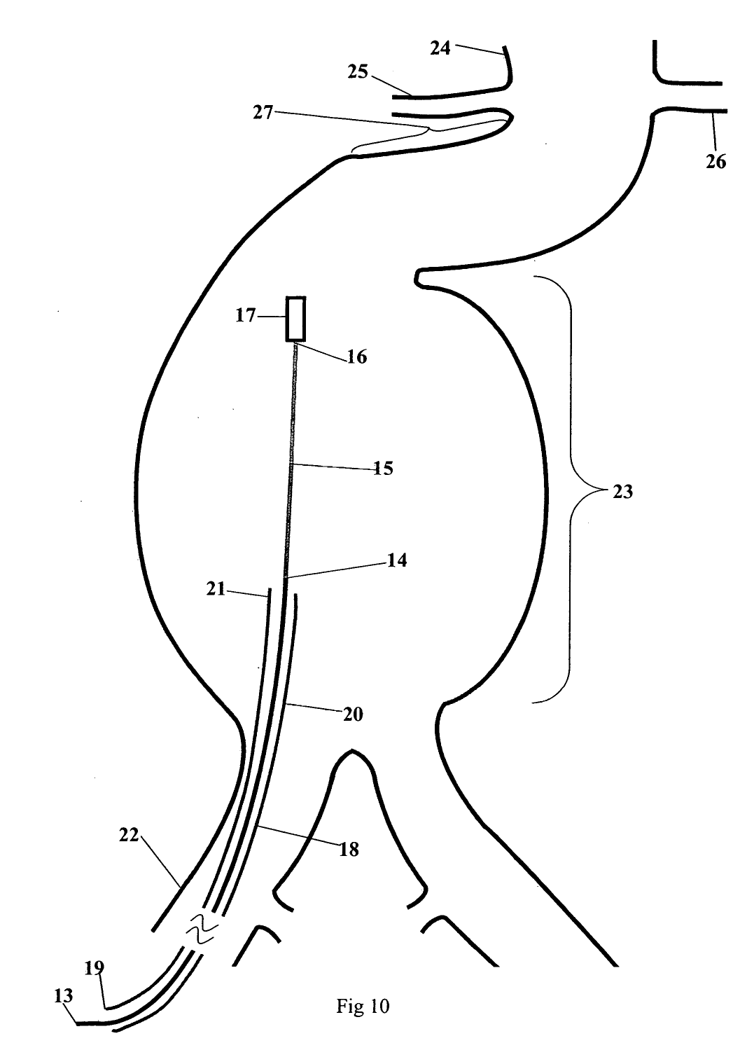

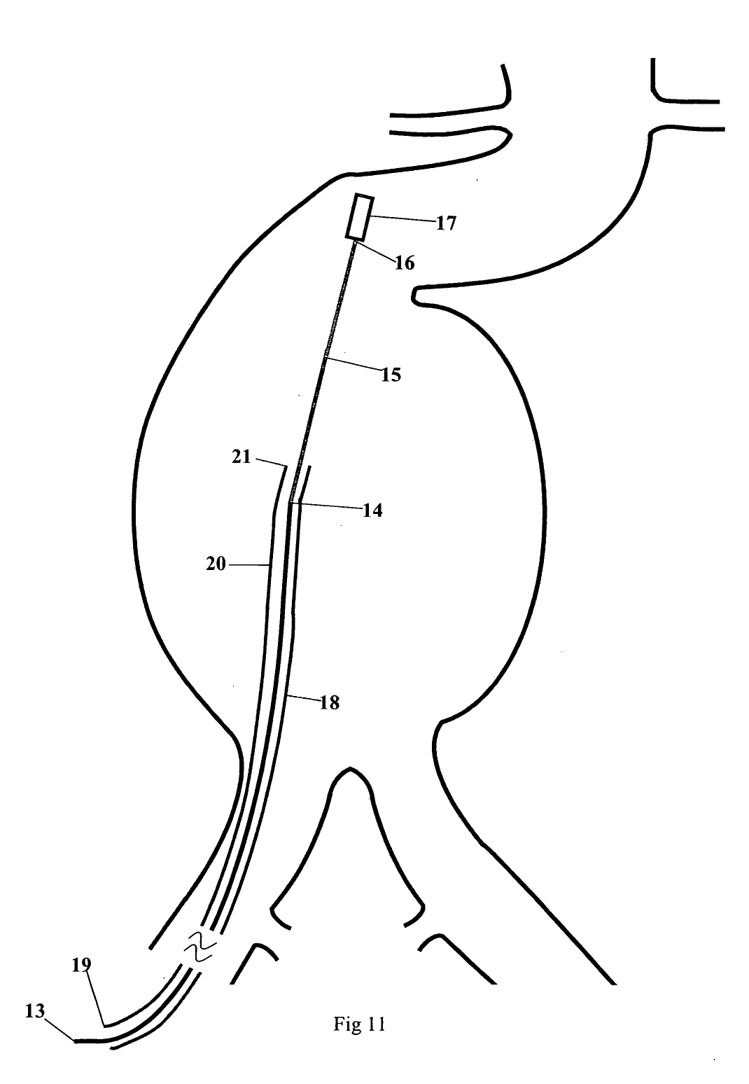

As shown in FIG. 5, delivery apparatus 12 comprising two or more segments, in this instance three segments 13-14, 14-15, and 15-16 of differing stiffness, is attached to surgical device 17 intended to be attached in part or in full, or manipulated, or both to or within a vessel. At least one of the distal segments (14-15 or 15-16) is more flexible than the proximal segment 13-14. The surgical device 17 is attached to the delivery apparatus 12, usually at the end 16 but it could be before the end as long as it is not attached to the proximal segment 13-14. As shown in FIG. 6, a catheter 6 is chosen with a straight portion 19-20 and a distal portion 20-21 that has a different (from 180 degrees) configuration on the end, in this example a curved portion 20-21.

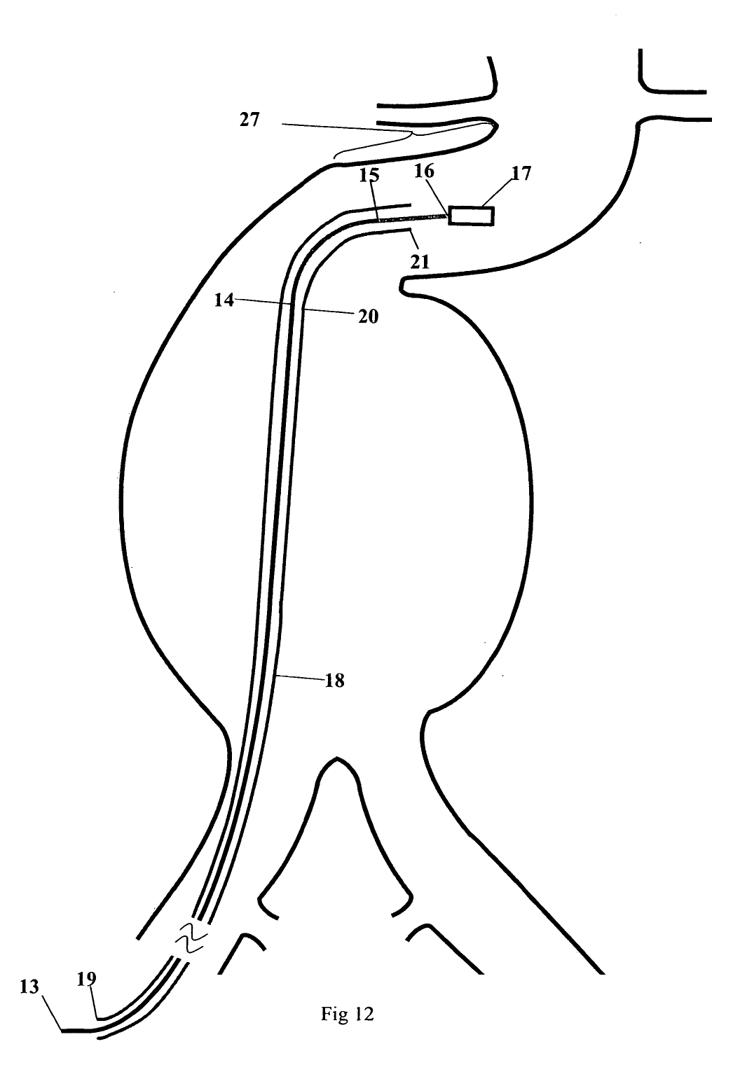

As shown in FIG. 7, delivery apparatus 12 with an attached surgical device 17 is inserted directly or within an introducer sheath into a vessel. The delivery apparatus 12, as noted above and depicted in FIG. 5, is composed of several segments 13-14, 14-15 and 15-16 of differing flexibility. Segments 13-14 and 14-15 can also be the same stiffness or 13-14 and 15-16 can be the same stiffness. Catheter 6, such as depicted in FIG. 6, can be chosen on the basis of its configuration or any other appropriate configuration. In this example, catheter 6 has the attribute of being more flexible than the most proximal segment 13-14 of the delivery apparatus 12. Thus, when catheter 6 is inserted over delivery apparatus 12 (FIG. 7) and advanced over the segment 13-14, catheter 6 is straightened out by the segment 13-14 of delivery apparatus 12 that has greater stiffness than the segment 20-21 of catheter 6 that has the different configuration. As catheter 6 is advanced over the next segment 14-15 (FIG. 8), this segment of delivery apparatus 12 is not as rigid as is the segment 20-21 of catheter 6 that has a curve. As a consequence, catheter 6 segment 20-21 begins to deflect segment 14-15 of delivery apparatus 12 that is not as stiff as catheter 6. When catheter 6 is advanced further (FIG. 9) over segment 14-15 of delivery apparatus 12, catheter 6 segment 20-21 deflects delivery apparatus 12 segment 14-15-16 with its attached surgical device 17 further into a right angle.

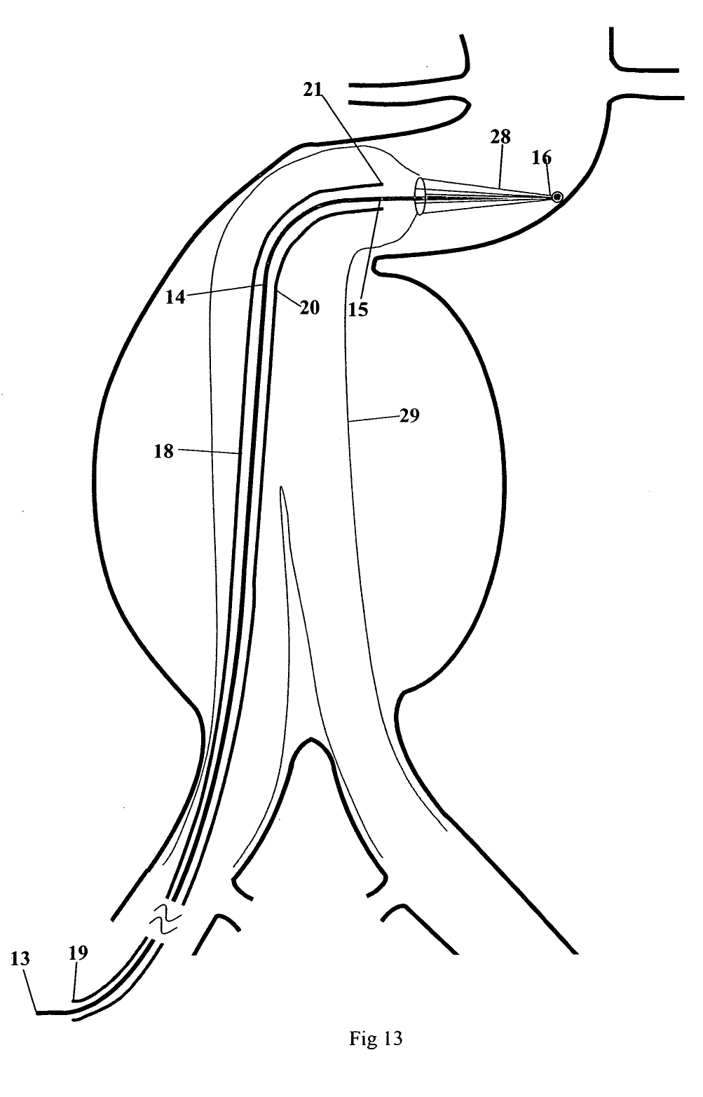

As an example of possible use, an embodiment to maneuver and attach part of surgical device 17 within the aorta of a patient with an abdominal aortic aneurysm. FIG. 10 depicts a catheter 6 inserted through the femoral/iliac artery 22 into the aneurysmal 23 portion of the aorta. Usually the suprarenal aorta 24 gives off a right 25 renal artery and a left 26 renal artery and continues through the abdomen at nearly 180 degrees. On occasion, however, the area just distal to the origins of the renal arteries that is not dilated 27 (this is usually termed the aortic neck) is angulated, making endovascular repair of the aneurysm difficult. The process described above may facilitate this type of repair. In FIG. 10, catheter 6 is passed up the rigid segment 13-14 of the delivery apparatus 12. Since the segment 13-14 of the delivery apparatus 12 is stiffer than the curved segment 20 21 of catheter 6, catheter 6 is kept at 180 degrees while on this portion of delivery apparatus 12. In FIG. 11, catheter 6 is advanced further onto segment 14-15 of delivery apparatus 12 that is not as stiff as catheter 6 curved segment 20-21. As a consequence catheter 6 begins to assume its preformed curved configuration and the more flexible segment 14-15 of delivery apparatus 12 begins to bend.

In FIG. 12, catheter 6 is advanced further such that the curved segment 20-21 of catheter 6 causes the less resistant segment 14-15 of delivery apparatus 12 to bend to an angle based on the preformed angle of catheter 6 making it possible to achieve placement of the surgical device 17 within the angulated aortic neck 27. This also allows for the cutomization of the catheter based on a particular patient's needs.

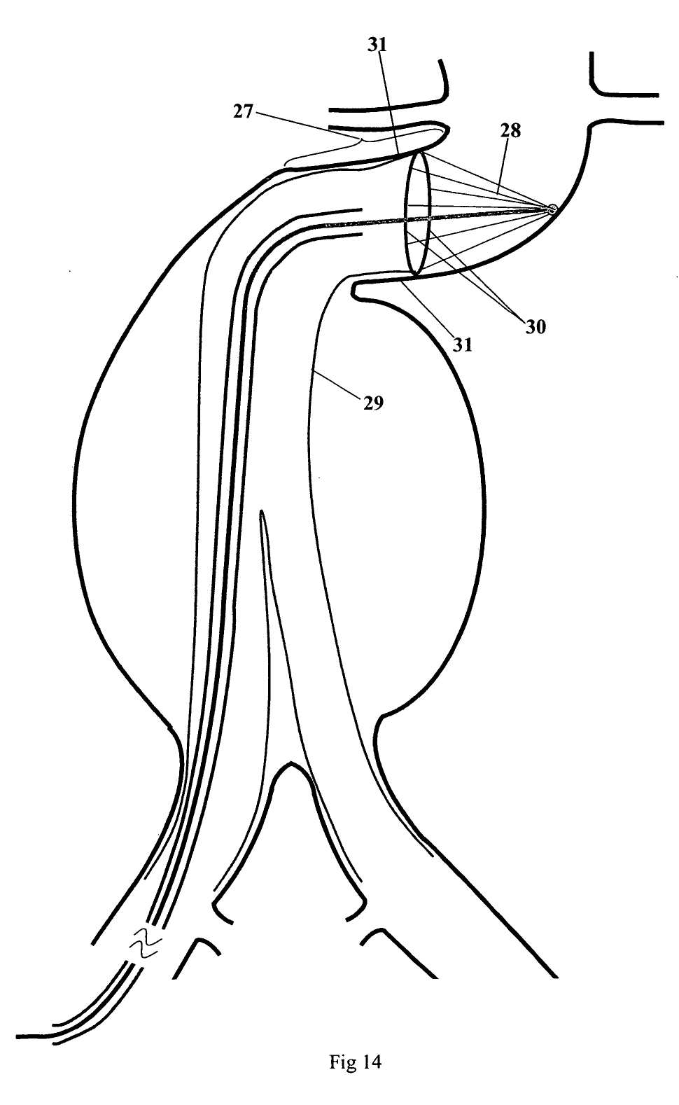

In one embodiment, surgical device 17 (FIGS. 7-12) can be substituted, as shown in FIG. 13, with an umbrella like support mechanism 28 attached to a prosthetic bifurcation graft 29 similar to what is used in the open operative procedure for repair of an abdominal aortic aneurysm, the utility of the ability to control angulation of surgical device 17 or, in this instance, the umbrella like suport mechanism 28, becomes more apparent. FIG. 14 shows the umbrella apparatus 28 in the open position such that the proximal portion 30 of the bifurcation graft 29 is held open against the aortic neck 27. In this way, surgical fasteners 105 can be used to attach the proximal portion 30 of the bifurcation graft 29 to the angulated aortic neck 27 in a circumfirential manner at 31.

Numerous characteristics and advantages have been set forth in the foregoing description, together with details of structure and function. The novel features are pointed out in the appended claims. The disclosure, however, is illustrative only, and changes, may be made in detail, especially in matters of shape, size, and arrangement of parts, within the principle of the invention, to the full extent indicated by the broad general meaning of the terms in which the appended claims are expressed.

1. A delivery apparatus for delivering a surgical device comprising;
a catheter having a distal segment, a proximate segment, and a lumen wherein the distal segment has a first configuration having a curved shape;
a delivery component within the lumen of the catheter having a first and a second segment wherein the first segment has rigidity greater than the catheter and the second segment has rigidity less than the catheter,
the distal segment having a second configuration that is straighter than the first configuration when the first segment of the delivery component is in communication with the lumen of the entire distal segment,
the distal segment having a third configuration having a curved shape when the second segment of the delivery component is in communication the lumen of the distal segment; and
a surgical device in communication with the delivery component.

2. The delivery apparatus of claim 1 wherein the delivery component is selected from the group consisting of a wire, a plastic rod, and a metal alloy tube.

3. The delivery apparatus of claim 2 wherein the delivery component is a plastic rod.

4. The delivery apparatus of claim 1 wherein the surgical device is in communication with the second segment of the delivery component.

5. The delivery apparatus of claim 1 wherein the first configuration is the same as the third configuration when the second component is in communication with the lumen of the entire distal end of the catheter.

6. The delivery apparatus of claim 1 wherein the delivery component further comprises of a third segment having rigidity between that of the first segment and the second segment wherein the distal segment has a fourth configuration having a degree of curvature between that of the second configuration and the third configuration when the third segment is in communication with the lumen of the distal segment of the catheter.

7. The delivery apparatus of claim 6 wherein the delivery component is selected from the group consisting of a wire, a plastic rod, and a metal alloy tube.

8. The delivery apparatus of claim 7 wherein the delivery component is a plastic rod.

9. The delivery apparatus of claim 6 wherein the surgical device is in communication with the second segment of the delivery component.

10. The delivery apparatus of claim 6 wherein the third segment is located between the first segment and the second segment of the delivery component.

11. The delivery apparatus of claim 6 wherein the first configuration is the same as the third configuration when the second component is in communication with the lumen of the entire distal end of the catheter.

12. A method for curving a distal end of a catheter having a lumen comprising,
providing the catheter, the distal end having a first configuration that is curved,
inserting a delivery component through the lumen, the delivery component having a first segment which is more rigid than the distal end of the catheter and is in communication with the lumen of the entire distal end of the catheter wherein the distal end assumes a second configuration that is substantially straight, and
moving the delivery component so that a second segment of the delivery component which is less rigid than the distal end of the catheter is in communication with the lumen of the distal end of the catheter wherein said distal end of the catheter assumes a third configuration that is curved.

13. The method of claim 12 wherein said first configuration is the same as the third configuration.

14. The method of claim 12 wherein said catheter is used to repair an abdominal aortic aneurysm.
